(19) United States
(12) Patent Application Publication
(10) Pub. No.: US 2005/0281866 A1
(43) Pub. Date: Dec. 22, 2005

(54) Adherent polymeric compositions

(75) Inventors: Peter K. Jarrett, Sudbury, MA (US); Kenneth A. Messier, Griswold, CT (US); Robert J. Miller, E. Bridgewater, MA (US); C. Michael Philbrook, Boston, MA (US); Hildegard M. Kramer, Westport, CT (US); J. Jeffrey Kablik, Tyngsboro, MA (US); Erika E. Johnston, Waltham, MA (US); Luis Z. Avila, Arlington, MA (US); Arthur J. Coury, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Genzyme Corporation, Cambridge, MA (US)

(21) Appl. No.: 11/136,328

(22) Filed: May 23, 2005

Related U.S. Application Data
(60) Provisional application No. 60/574,111, filed on May 24, 2004.

(51) Int. Cl. A61L 15/16

(52) U.S. Cl. 424/448

(57) ABSTRACT

Described herein are adhesive polymeric compositions and methods for using the compositions. The composition are adherent to the applied surface. The compositions, in certain embodiments, are biodegradable and biocompatible, and can be designed with selected properties of compliancy and elasticity for different surgical and therapeutic applications. The adherent polymeric compositions comprise a polymerized macromer network and an additive mixed or entangled in the polymerized macromer. The additive is bonded to a surface by at least one covalent bond or by secondary interactions and is not covalently bonded to the polymerized macromer network. Alternatively, the additive is bonded to the surface by at least one covalent bond and is also bonded to the macromer network. The disclosed compositions can be used as an improved barrier, coating or drug delivery system that due to the additive is highly adherent to an applied surface. The compositions of the present invention are typically non-toxic, water miscible and have adaptable characteristics depending on the macromers and additives used. For example, specific macromers can be used for targeted bioresorption rate and/or degradation rate of the applied composition.

### RELATED APPLICATION

This application claims the benefit of U.S. Provisional Application No. 60/574,111, filed on May 24, 2004. The entire teachings of the above application are incorporated herein by reference.

### BACKGROUND OF THE INVENTION

Polymers have been used extensively by researchers for coating of surfaces in a living organism. Locally polymerized gels have been used as barriers and drug delivery devices for several medical conditions. For example, Hubbell et al, (U.S. Pat. No. 5,410,016) describe two methods for photopolymerization to form a gel. However, adherence of the formed gel to the surface can be a problem, especially under surgical conditions where the tissue surface to be treated is wet and may further be covered with blood, mucus or other secretions. Previous methods of application, such as those requiring priming of the tissue, can be complex, cumbersome, and lengthy and thus ill-suited for delicate or endoscopic surgical procedures.

Although the surface adhering materials of the prior art are suitable for application to tissue and other substrates, adherence is in many cases limited and in certain cases essentially non-existent. Thus, there is a need for compositions and methods for coating surfaces with improved adherence that are biocompatible, do not elicit specific or non-specific immune responses, and which can be polymerized in contact with living cells or tissue without injuring or killing the cells, within a very short time frame, and with varying thicknesses. Important criteria for the use of these adherent compositions in vivo is that they should be biodegradable and able to rapidly polymerize within the time of a short surgical procedure.

### SUMMARY OF THE INVENTION

It has been found that the compositions as described herein are useful as adherent polymeric compositions for coatings of surfaces, in particular tissue surfaces. Applications for the compositions include prevention of adhesion formation after surgical procedures, controlled release of drugs and other bioactive species, temporary protection or separation of tissue surfaces, adhering or sealing tissues together, sealing barrier against leaks of body fluid or gas from a tissue or vessel, filling or bulking of tissue defects or other tissue repair, and preventing the attachment of cells to tissue surfaces.

The present invention provides adhesive polymeric compositions, comprising a polymerized macromer network and an additive mixed or entangled in the polymerized macromer. The additive is bonded to a surface by at least one covalent bond or by secondary interactions and is not covalently bonded to the polymerized macromer network. Preferably, the additive is covalently bonded to the surface by at least one covalent bond, wherein the covalent bond is formed by the reaction between a functional group (hereinafter “surface reactive group”) on the additive and a functional group on the surface (hereinafter “additive reactive group”). The additive provides increased adherent characteristics to the macromer network as compared to conventional macromer networks without the additive.

In another aspect, the adhesive polymer composition comprise a polymerized macromer network and an additive mixed or entangled in the polymerized macromer. The additive is bonded to a surface by at least one covalent bond formed by the reaction between a functional group on the additive (hereinafter “Functional Group A) and a functional group on the surface; and the additive is bonded to the macromer network by at least one covalent bond formed by the reaction between a functional group on the additive (hereinafter “Functional Group B”) and a functional group on the macromer network. Functional group A is non-reactive with the macromer network and Functional Group B is non-reactive with the surface.

In another aspect of the invention, the composition can optionally include a top layer of a second polymerized macromer wherein the first polymerized macromer is bonded to the second polymerized macromer.

Also described herein are polymeric compositions for adhering to a surface, comprising: a) a first solution of at least one polymerizable macromer; b) an additive having at least one surface reactive group and wherein the additive is non reactive with the macromer; and c) a polymerization initiator or a first and second agent that when combined reacts to form a polymerization initiator. The polymerization of the macromer of a) is initiated by the initiator of c). The additive comprises a surface reactive group at each terminus that can bind to the surface covalently.

In other embodiments, the composition further comprises a second solution of the second agent and wherein the first agent is dissolved in the first solution. The first and second agents form a reduction/oxidation (redox) reaction when combined. In certain embodiments, the second solution comprises at least one polymerizable macromer polymerizable with the macromer of the first solution.

In yet another embodiment, polymeric compositions are described for adhering to a surface, comprising a) a polymerizable macromer b) an additive comprising at least one Functional Group A and at least one Functional Group B, wherein Functional Group A can react with a functional group on the surface to form a covalent bond with the surface but is non-reactive with the polymerizable macromer and wherein Functional Group B can form a covalent bond with a functional group on the polymerizable macromer to form a covalent bond with the polymerizable macromer but is non-reactive with the surface; and c) a polymerization initiator or a first and second agent that when combined reacts to form a polymerization initiator, wherein polymerization of the macromer of a) is initiated by the initiator of c).

Also described are methods of coating a surface, comprising: a) applying to the surface an additive capable of binding to the surface and a polymerizable macromer; b) binding said additive to the surface; and c) polymerizing said macromer, thereby entangling the additive with the polymerized macromer. In certain methods, the macromer and additive are simultaneously applied to the surface. In certain embodiments, the polymerization of the macromer is initiated prior to applying to the surface, while the macromer is being applied to the surface or after applying the macromer to the surface. The additive is a polymer that binds to the surface through a covalent bond at one or more terminus. In certain aspects, the additive and the macromer are added together and a top coat of a second polymerizable macromer is applied after step b) and polymerized.

Also described are methods of adhering a polymeric composition to a surface, comprising: a) applying an additive and a polymerizable macromer to the surface, wherein the additive comprises at least one Functional Group A and at least one Functional Group B, wherein Functional Group A can react with a functional group on the surface to form a covalent bond with the surface but is non-reactive with the polymerizable macromer and wherein Functional Group B can form a covalent bond with a functional group on the polymerizable macromer to form a covalent bond with the polymerizable macromer (or polymerized macromer) but is non-reactive with the surface; b) polymerizing the macromer to form a polymerized macromer network; and c) reacting Functional Group A with the surface, reacting Functional Group B with the polymerizable macromer or polymerized macromer network.

### BRIEF DESCRIPTION OF THE DRAWINGS

FIG. 1 is a schematic drawing of a composition encompassed by the instant invention depicting the covalent binding of an additive (PEG-dialdehyde) to the surface entrapped in the network (PEG-macromer).

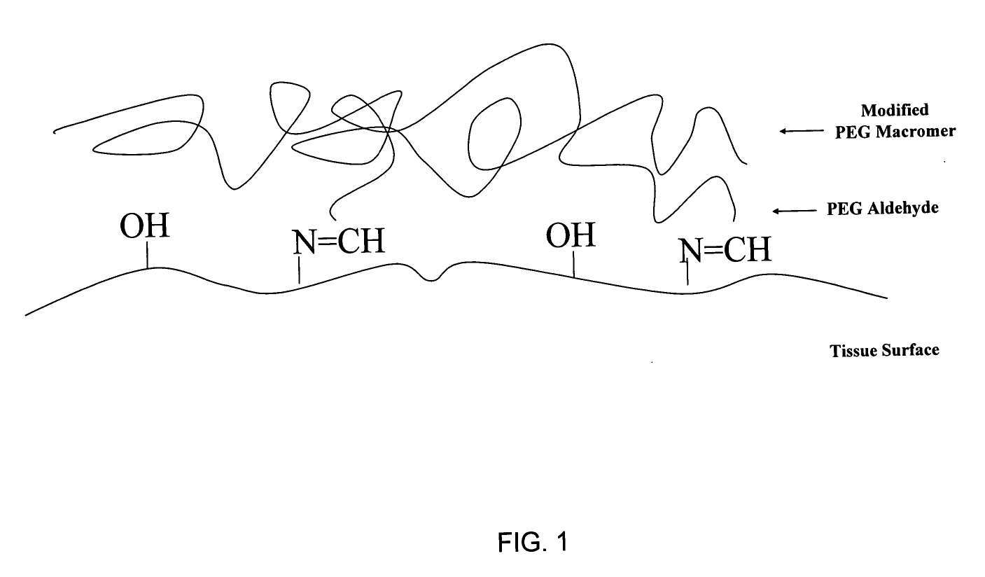

FIG. 2 depicts two scenarios, Group I and Group II. Group I shows the surface reactive groups in the additive that are possible in combination with the polymerizable groups in the macromer (shown with an “x”). In Group I, the tissue reactive groups do not react with the polymerizable groups on the macromer. Group II shows the possible network reactive groups in the additive for combination with the polymerizable groups on the macromer (shown with an “x”). In Group II, the network reactive groups react with the polymerizable groups on the macromer.

### DETAILED DESCRIPTION OF THE INVENTION

A description of preferred embodiments of the invention follows.

The present invention provides methods and compositions with improved adherence of the compositions to surfaces, particularly tissue surfaces of a patient or surfaces of medical devices for temporary or permanent implantation into a patient. Further, methods are described for applying the compositions to a surface, for example the tissue surface of a patient. “Patient” as that term is used herein refers to the recipient of the compositions and methods described herein. Mammalian and non-mammalian patients are included. In certain embodiments, the patient is a mammal, such as a human, canine, murine, feline, bovine, ovine, swine or caprine. In a preferred embodiment, the patient is a human.

The adherent polymeric compositions comprise a polymerized macromer network and an additive mixed or entangled in the polymerized macromer. The additive is bonded to a surface by at least one covalent bond or by secondary interactions and is not covalently bonded to the polymerized macromer network. Alternatively, the additive is bonded to the surface by at least one covalent bond and is also bonded to the macromer network. The disclosed compositions can be used as an improved barrier, coating or drug delivery system that due to the additive is highly adherent to an applied surface. The compositions of the present invention are typically non-toxic, water miscible and have adaptable characteristics depending on the macromers and additives used. For example, specific macromers can be used for targeted bioresorption rate and/or degradation rate of the applied composition.

The components of the compositions are described in detail below.

Additive

In the disclosed adhesive polymeric compositions, an “additive” is a component that is separate from the polymerized macromers network or polymerizable macromer composition and is able to adhere to the surface. In addition, the additive becomes entangled with the polymerized macromer network upon polymerization of the macromer. Because of its entanglement in the network and its adherence to the surface, an additive in the disclosed compositions acts to keep the network in contact with or in close proximity to surface.

The additives of the disclosed compositions are bonded (or capable of bonding) to a surface by at least one covalent bond and/or by secondary interactions (e.g., ionic bonding, hydrogen bonding, van der Waals forces, hydrophobic interactions, and the like), typically by at least one covalent bond that is formed by a reaction between a functional group on the additive and a functional group on the surface. It is noted that in the free radical polymerization of a polymerizable macromer with free radical polymerizable groups, it is possible that a small percentage of polymerizable macromers may react with free radicals on a surface. Polymerizable macromers of this type are not considered to be an “additive” because, for example, the covalent bond between the additive and the surface must be formed between a functional group on the additive and a functional group on the surface and not a free radical that forms on the surface.

“Entangled within the polymerized macromer network” means that the additive is surrounded by and intertwined with the network. More specifically, the additive is interwoven, at least in part, within the network, analogous to the entanglement of a string within a three dimensional wire mesh. Thus, if the two ends of at least some of the additive molecules emerge from the network and are bound to a surface, entanglement of the center portion of these molecules in the network acts to keep the network in contact with or in close proximity to the surface. Even if only one end of the additive is bonded to the surface, the entanglement of the additive in the network will resist separation of the network from the surface. Thus, the additive is a substantially linear molecule that is of sufficient length to become entangled within the polymerized macromer network. Substantially linear means single-strand chains as well as branched or multi-armed chains such that the additive has two or more termini.

In one embodiment of the disclosed compositions, there are no covalent bonds between the additive and the polymerized macromer network (or the additive is non-reactive with the polymerizable macromer). Typically, additives which are non-reactive with the polymerizable macromers contain two or more reactive functional groups (hereinafter “surface reactive groups”) that are capable of reacting with functional groups on the surface (or which react to form a covalent bond with functional groups on the surface). Most commonly, a reactive group is at each terminus of the additive. Surface reactive groups are therefore “orthogonal” to, i.e., non-reactive with, polymerizable groups on the polymerizable macromers. When the polymerizable macromers comprise free radical polymerizable functional groups, the surface reactive groups are not typically reactive to free radical polymerization. When the surface reactive groups are nucleophilic, then the reactive functional groups on the surface are electrophilic, and vice versa. Reactive functional groups on a surface are typically nucleophilic, e.g., hydroxyl, amines and thiols. Commonly used surface reactive groups for covalent bonding to tissue surfaces include aldehyde, N-hydroxysuccinimide ester and related active esters, maleimide, isocyanate, disulfide, epoxide, aziridine, carbodiimide, episulfide, ketene, carboxylate, phosphate, alkyl halides (alkylating agents) and alkyl sulfonate esters (alkylating agents).

In another embodiment of the disclosed compositions, the additive is entangled within the polymer network, is covalently bonded to the surface (or is capable of binding to the surface) and is covalently bonded to the network (or is capable of covalently binding to the polymerizable macromer). The covalent bond to the surface is formed by a reaction between a functional group on the additive (a surface reactive group) and a functional group on the surface. The surface reactive group is non-reactive with the network or the polymerizable macromer. Therefore, if the polymerizable macromer contains a free radical polymerizable group, the surface reactive group is non-reactive during radical polymerization. Commonly used surface reactive groups for reaction with tissue surfaces are as described in the previous paragraph. The covalent bond between the additive and the macromer network is formed by a reaction between a functional group on the additive different from the surface reactive groups (hereinafter a “network reactive group”) and a functional group on the polymerizable macromer. In certain embodiments, the network reactive groups include, thiol, ethylenecally unsaturated groups and acetyleneically unsaturated groups. In certain embodiments, the polymerizable group on the polymerizable macromer and the network reactive group on the additive are the same. In a preferred embodiment, both groups are ethylenically unsaturated groups such as described in more detail below.

As noted above, the additive is substantially linear, although it may be multi-armed or branched and is of sufficient size to become entangled with the polymerized macromer network. The properties of the composition can be modified by appropriate selection of the additive, e.g., by the reactive functional groups on the additive, the size of the additive and the nature of the polymeric backbone between the functional groups. Typically, the additive has a molecular weight between 1,000 Daltons and 100,000 Daltons; more commonly between 3,000 Daltons and 35,000 Daltons. Often, the additive is a linear polymer with reactive functional groups at each terminus. When used with tissue surfaces, the additive should be biocompatible. Hydrophilic polymers such as polyalkyleneglycols, poly(ethylene glycol), poly(ethylene oxide), partially or fully hydrolyzed poly(vinyl acetate) or fully hydrolyzed polyvinyl acetate (also referred to as polyvinyl alcohol), poly(vinylpyrrolindone), poly(ethyloxazoline), poly(ethylene oxide)-co-poly(proplylene oxide) block copolymers (poloxamers and meroxapols) poloxamines, carboxymethyl cellulose, hydroxyalkylated cellusoses such as hydroxyethyl cellulose and methylhydroxyproply cellulose, polyesters, polypeptides, polynucleotides, polysaccharides or carbohydrates such as Ficoll®, polysucrose, hyaluronic acid, dextran, heparin sulfate, chondroitin sulfate, heparin, or alginate, and proteins such as gelatin, collagen, albumin, or ovalbumin are commonly used. Polyethyleneglycols are preferred. Examples of suitable additives and their use for binding to tissue surfaces are disclosed in U.S. Pat. No. 5,977,252 to Wagner, et al., U.S. Pat. No. 5,936,035 to Rhee et al., and U.S. Pat. No. 6,566,406 to Pathak et al., the entire teachings of which are incorporated herein by reference.

Macromers

A macromer, as defined herein, is a polymeric molecule which can be reacted to form a network. In preferred embodiments, the macromer is a polymer that can be reacted, for example polymerized, to form a network. A network is a crosslinked polymer. A network can be either a linear polymer with crosslinking groups or a polymerized crosslinked macromer formed when at least some of the polymerizable macromers comprise more than one polymerizable group. The macromer network can also be non-linear, for example star-shaped or dendritic-shaped.

A polymerizable macromer comprises at least one polymerizable group effective as a crosslinker, so that the macromers can be crosslinked to form a network. The minimal proportion required will vary with the nature of the macromer and its concentration in solution. For example, the macromers should include at least one polymerizable group on average, and, preferably, the macromers each include two or more polymerizable groups on average.

A polymerizable group is a reactive functional group that can form covalent bonds resulting in a polymerized macromer network. Suitable groups include, but are not limited to, ethylenically or acetylenically unsaturated groups, (aliphatic hydroxy groups are not considered to be reactive groups for the chemistry disclosed herein, except in formulations which also contain groups capable of covalent crosslinking with such hydroxyls.) Ethylenically unsaturated groups include vinyl groups such as vinyl ethers, N-vinyl amides, allyl groups, unsaturated monocarboxylic acids, unsaturated dicarboxylic acids, and unsaturated tricarboxylic acids. Unsaturated monocarboxylic acids include acrylic acid, methacrylic acid and crotonic acid. Unsaturated dicarboxylic acids include maleic, fumaric, itaconic, mesaconic or citraconic acid. Unsaturated tricarboxylic acids include aconitic acid. Polymerizable groups may also be derivatives of such materials, such as acrylamide, N-isopropylacrylamide, hydroxyethylacrylate, hydroxyethylmethacrylate, and analogous vinyl and allyl compounds. Reactive group forming compounds will preferably be available in a stable activated form, to allow simple incorporation into the macromer. Examples of such materials are (meth)acrylyl chloride, acrylic anhydride, and allyl glycidyl ether. The polymerizable groups can be located within the macromer. In another aspect, the polymerizable groups are located at one or more ends of the macromer.

In polymerization, each polymerizable group will polymerize into a chain and crosslinked networks can be produced using only slightly more than one polymerizable group per macromer (i.e., about one polymerizable groups on average). However, higher percentages are preferable, and networks can be obtained in polymer mixtures in which most or all of the molecules have two or more reactive double bonds. Poloxamines, an example of a non-linear hydrophilic block, have four arms and thus may readily be modified to include four polymerizable groups.

Polymerization is initiated by any convenient reaction, including photopolymerization, chemical or thermal free-radical polymerization, redox reactions, cationic polymerization, and other chemical reactions of active groups which are orthogonal to the tissue reactive group of the additive. Initiators for use in the polymerization reaction are discussed in detail below.

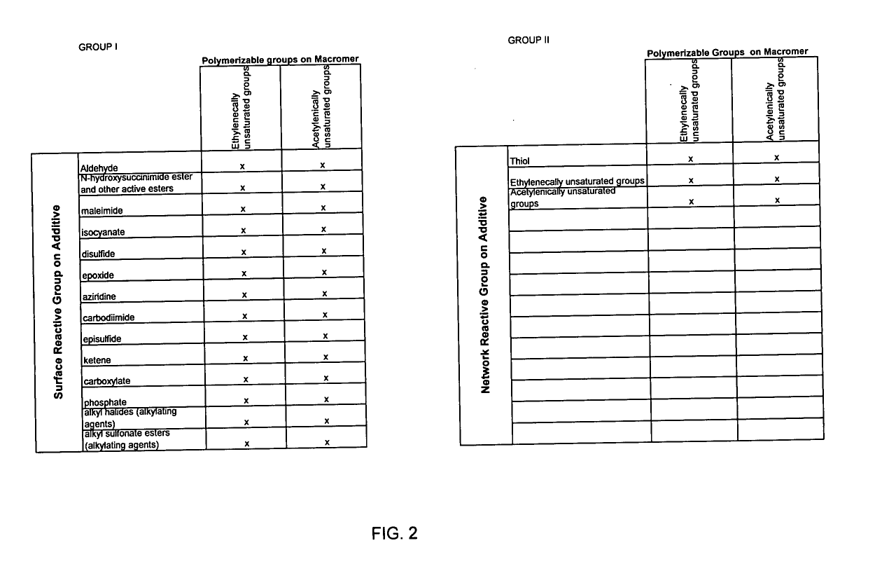

FIG. 2 shows possible combinations of the polymerizable groups of the macromer and the surface reactive groups on the additive for Group I, when the tissue reactive group on the additive does not react with the macromer, and Group II, when the polymerizable group on the macromer reacts with the network reactive group on the additive.

Types of Macromers

The monomers for use in the macromers may be large molecules containing polymerizable groups, such as acrylate-capped polyethylene glycol (PEG-diacrylate), or other polymers containing ethylenically-unsaturated groups, such as those of U.S. Pat. No. 4,938,763 to Dunn et al, U.S. Pat. Nos. 5,100,992 and 4,826,945 to Cohn et al, U.S. Pat. Nos. 4,741,872 and 5,160,745 to De Luca et al., U.S. Pat. No. 5,410,016 by Hubbell et al, U.S. Pat. No. 6,177,095 to Sawhney et al., or U.S. Pat. No. 6,201,065 to Pathak et al., the entire teachings of which are incorporated herein by reference. The molecular weight of the larger molecules is preferably between about 3,000 Daltons and about 1,000,000 Daltons, more preferably, between about 10,000 Daltons and about 35,000 Daltons, and most preferably, between about 3,000 Daltons and about 10,000 Daltons.

Properties of the macromer, other than polymerizability, will be selected according to the type of use, following principles known in the art. There is extensive literature on the formulation of polymerizable coating materials for particular applications; these formulae can readily be adapted to use the improved adherence-promoting polymerization system described herein with little experimentation.

Preferred Macromers

The properties of the macromers of the composition can be modified by appropriate selection of the polymer core, polymerizable groups and optional functional groups that can be configured within the macromer in a variety of ways for producing various macromer compositions.

The non-polymerizable portion of the macromer is referred to herein as its “core”. Examples of core polymers include polyamino acids, polyalkylene glycols, polysaccharides and the like. The molecular weight of the polymer can vary depending on the desired application. In most instances, the weight average molecular weight is about 100 Daltons to about 2,000,000 Daltons, preferably about 1,000 Daltons to 1,000,000 Daltons, more preferably about 1,000 Daltons to about 100,000 Daltons, and most preferably about 3,000 Daltons to about 35,000 Daltons. When the polymer core is polyethylene glycol, the preferred molecular weight is in the range of about 1,000 Daltons to about 40,000 Daltons, preferably between 3,000 Daltons about 30,000 Daltons.

A basic macromer for use in the compositions and methods described herein minimally comprises a polymer core with polymerizable groups. This polymer core structure can then be adapted in many ways by the addition of various groups within the polymer structure. These groups include one or more hydrophilic blocks, and one or more biodegradable blocks. In preferred embodiments, the macromers are block copolymers cores that further include a biodegradable block, a hydrophilic block, and at least one polymerizable group at each terminus, such as an acrylate group. By varying the number, size, positions and types of groups, the macromer can have variety of properties and characteristics as will be discussed in detail below.

Hydrophilic Block

The hydrophilic blocks, as used herein, can be a single block with a molecular weight of at least 600, preferably 2000 or more, and more preferably at least 3000 Daltons. Alternatively, the hydrophilic blocks can be two or more hydrophilic blocks which are joined by other groups. Such joining groups can include biodegradable linkages, polymerizable linkages, or both. For example, an unsaturated dicarboxylic acid, such as maleic, fumaric, or aconitic acid, can be esterified with biodegradable groups as described below, and such linking groups can be conjugated at one or both ends with hydrophilic groups such as polyethylene glycols.

Water-soluble hydrophilic oligomers may be incorporated into the biodegradable macromers. Suitable hydrophilic (also referred to as water-soluble) polymeric blocks include those prepared from poly(ethylene glycol), poly(ethylene oxide), partially hydrolyzed poly(vinyl acetate) or fully hydrolyzed poly vinyl acetate (also referred to as polyvinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, hydroxyalkylated celluloses such as hydroxyethyl cellulose and methylhydroxypropyl cellulose, polypeptides, polynucleotides, polysaccharides or carbohydrates such as Ficoll®, polysucrose, hyaluronic acid, dextran, heparan sulfate, chondroitin sulfate, heparin, or alginate, and proteins such as gelatin, collagen, albumin, or ovalbumin. Preferably, the water-soluble polymeric blocks are made from poly(ethylene glycol) or poly(ethylene oxide).

The hydrophilic polymer blocks may be intrinsically biodegradable or may be poorly biodegradable or effectively non-biodegradable in the body. In the latter two cases, the blocks should be of sufficiently low molecular weight to allow excretion. The maximum molecular weight to allow excretion in human beings (or other species in which use is intended) will vary with polymer type, but will often be about 50,000 Daltons or below. Hydrophilic natural polymers and synthetic equivalents or derivatives, including polypeptides, polynucleotides, and degradable polysaccharides, can be used.

Biodegradable Blocks

The biodegradable blocks are typically hydrolyzable under in vivo conditions. In certain aspects of the invention, the biodegradable block comprises one or more units of carbonates, such as formed from trimethylcarbonate, esters, such as glycolate or lactate moieties, dioxanone, orthoesters, anhydrides, or other synthetic or semisynthetic degradable linkages. Natural materials may be used in the biodegradable sections when their degree of degradability is sufficient for the intended use of the macromer. Such biodegradable blocks may comprise natural or unnatural amino acids, carbohydrate residues, and other natural linkages. Biodegradation time will be controlled by the local hydrolytic degradation or enzymatic hydrolysis. The availability of such enzymes may be ascertained from the art or by routine experimentation.

Additional biodegradable blocks can include polymers or copolymers and oligomers or cooligomers of hydroxy acids or other biologically degradable polymers that yield materials that are non-toxic or present as normal metabolites in the body. Preferred poly(hydroxy acid)s are poly(glycolic acid), poly(D, L-lactic acid) and poly(L-lactic acid). Other useful materials include poly(amino acids), poly(anhydrides), poly(orthoesters), and poly(phosphoesters). Polylactones such as poly(epsilon-caprolactone), poly(delta-valerolactone), poly(gamma-butyrolactone) and poly (beta-hydroxybutyrate), for example, are also useful.

Biodegradable regions can be constructed from monomers, oligomers or polymers using linkages susceptible to biodegradation, such as ester, peptide, anhydride, orthoester, and phosphoester bonds. The biodegradable group can be an oligomer comprsing one or more carbonate or dioxanone linkages.

By varying the total amount of biodegradable blocks, and selecting the ratio between the number of carbonate or dioxanone linkages (which are relatively slow to hydrolyze) and of lower hydroxy acid linkages (especially glycolide or lactide, which hydrolyze relatively rapidly), the degradation time of the adherent polymeric compositions formed from the macromers can be controlled.

In preferred embodiments, at least one biodegradable region is a carbonate, or dioxanone linkage. In a most preferred embodiment, at least one biodegradable region is trimethylcarbonate or lactate.

Any carbonate can be used in the macromers described above. In certain embodiments, carbonates are aliphatic carbonates, for maximum biocompatibility. For example, trimethylene carbonate and dimethyl carbonate are examples of aliphatic carbonates. Lower dialkyl carbonates are joined to backbone polymers by removal by distillation of alcohols formed by equilibration of dialkyl carbonates with hydroxyl groups of the polymer.

In other embodiments, cyclic carbonates, which can react with hydroxy-terminated polymers without release of water are used. Suitable cyclic carbonates include ethylene carbonate (1,3-dioxolan-2-one), propylene carbonate (4-methyl-1,3-dioxolan-2-one), trimethylene carbonate (1,3-dioxan-2-one) and tetramethylene carbonate (1,3-dioxepan-2-one). Under some reaction conditions, it is possible that orthocarbonates may react to give carbonates, or that carbonates may react with polyols via orthocarbonate intermediates, as described in Timberlake et al, U.S. Pat. No. 4,330,481. Thus, certain orthocarbonates, particularly dicyclic orthocarbonates, can be suitable starting materials for forming the carbonate-linked macromers as used in the compositions described herein.

Alternatively, suitable diols or polyols, including backbone polymers, can be activated with phosgene to form chloroformates, as is described in the art, and these active compounds can be mixed with backbone polymers containing suitable groups, such as hydroxyl groups, to form macromers containing carbonate linkages. All of these materials are “carbonates” as used herein.

Suitable dioxanones include dioxanone (p-dioxanone; 1,4-dioxan-2-one; 2-keto-1,4-dioxane), and the closely related materials 1,4-dioxolan-2-one, 1,4-dioxepan-2-one and 1,5-dioxepan-2-one. Lower alkyl, for example C1-C4 alkyl, derivatives of these compounds are also contemplated, such as 2-methyl p-dioxanone (cyclic O-hydroxyethyl ether of lactic acid).

In certain aspects of the invention, the macromers contain between about 0.3% and 20% by weight of carbonate residues or dioxanone residues, or, between about 0.5% and 15% carbonate or dioxanone residues, or, about 1% to 5% carbonate or dioxanone residues. In those embodiments where hydroxy acid residues are desired, the macromer contains between about 0.1 and 10 residues per residue of carbonate or dioxanone, more preferably between about 0.2 and 5, and most preferably one or more such residue per macromer.

In general, any formulation of the macromer that is intended to be biodegradable must be constructed so that each polymerizable group is separated from the other polymerizable group by one or more linkages that are biodegradable. Non-biodegradable materials are not subject to this constraint.

Copolymer Cores

In certain embodiments, the macromer includes a copolymer core capped at least one terminus with a polymerizable group, such as an acrylate group. The copolymer core is a multi-block copolymer of a hydrophilic polymer and biodegradable groups or oligomers of biodegradable groups. Subsequently, each terminus of the copolymer core is modified to comprise a polymerizable group capable of cross-linking the resulting macromers.

The individual polymeric blocks can be arranged to form different types of block copolymers, including di-block, tri-block, and multi-block copolymers. The polymerizable groups can be attached directly to biodegradable blocks or indirectly via water-soluble nondegradable blocks, and are preferably attached so that the polymerizable groups are separated from each other by a biodegradable block. For example, if the macromer contains a hydrophilic block coupled to a biodegradable block, one polymerizable group may be attached to the hydrophilic block and another attached to the biodegradable block. Preferably, both polymerizable groups would be linked to the hydrophilic block by at least one biodegradable linkage.

The di-block copolymers include a hydrophilic block linked to a biodegradable block, with one or both terminals capped with a polymerizable group. The tri-block copolymers can include a central hydrophilic block and outside biodegradable blocks, with one or both terminals capped with a polymerizable group. Alternatively, the central block can be a biodegradable block, and the outer blocks can be hydrophilic. The multiblock copolymers can include one or more of the water-soluble blocks and biocompatible blocks coupled together in a linear fashion. Alternatively, the multiblock copolymers can be brush, comb, dendritic or star copolymers. If the backbone is formed of a water-soluble block, at least one of the branches or grafts attached to the backbone is a biodegradable block. Alternatively, if the backbone is formed of a biodegradable block, at least one of the branches or grafts attached to the backbone is a hydrophilic block, unless the biodegradable block is also hydrophilic.

The individual polymeric blocks may have uniform compositions, or may have a range of molecular weights, and may be combinations of relatively short chains or individual species which confer specifically desired properties on the final composition, while retaining the required characteristics of the macromer. The lengths of oligomers referred to herein may vary from single units (in the biodegradable portions) to many, subject to the constraint of preserving the overall water-solubility of the macromer. For example, the macromers that polymerized to make the network comprise block copolymer cores that further include a biodegradable group, a hydrophilic block, and at least one polymerizable group at each terminus, such as an acrylate group.

In certain embodiments, the core includes hydrophilic poly(ethylene glycol) oligomers of molecular weight between about 400 Daltons and 40,000 Daltons; biodegradable blocks, such as poly (α-hydroxy acid) oligomers of molecular weight between about 200 Daltons and 1200 Daltons; and optionally oligomers of trimethylene carbonate or dioxanone. The core is then capped at each terminus with an acrylate-type monomer or oligomer (i.e., containing carbon-carbon double bonds) of molecular weight between about 50 Daltons and 200 Daltons which are capable of cross-linking and polymerization between the resulting macromers. More specifically, a preferred embodiment incorporates a core consisting of poly(ethylene glycol) oligomers of molecular weight between about 8,000 Daltons and 20,000 Daltons; biodegradable groups comprising poly(lactic acid) oligomers of average molecular weight about 150 to 500 Daltons and optionally oligomers of trimethylene carbonate of average molecular weight of about 500 to 1000 Daltons; and capped terminus of acrylate moieties of about 100 Daltons molecular weight.

Those skilled in the art will recognize that oligomers of the hydrophilic block, biodegradable group and polymerizable group on the terminus may have uniform compositions or may be averages of combinations of relatively short chains or individual species which confer specifically desired properties on the final composition while retaining the specified overall characteristics of each section of the macromer. The lengths of oligomers referred to herein may vary from two mers to many and usually corresponds to average lengths due to random oligomerization, the term being used to distinguish subsections or components of the macromer from the complete entity.

In the particular application area of coating or applying to tissues, cells, medical devices, and capsules, formation of implants for drug delivery or as mechanical barriers or supports, and other biologically related uses, the general requirement of the compositions include biocompatibility and lack of toxicity. For all biologically-related uses, toxicity must be low or absent in the finished state for externally coated non-living materials, and at all stages for internally-applied materials. Biocompatibility, in the context of biologically-related uses, is the absence of stimulation of a severe, long-lived or escalating biological response to an implant or coating, and is distinguished from a mild, transient inflammation which accompanies implantation of essentially all foreign objects into a living organism.

The macromer solutions in general should not contain harmful or toxic solvents. In certain aspects, the monomers are substantially soluble in water to allow their application in a physiologically-compatible solution, such as buffered isotonic saline. Water-soluble coatings may form thin films, but more preferably form three-dimensional gels of controlled thickness.

In cases involving implants, the adherent polymeric compositions formed should be biodegradable, so that it does not have to be retrieved from the body. Biodegradability, in this context, is the predictable disintegration of an implant into small molecules which will be metabolized or excreted, under the conditions normally present in a living tissue.

Elasticity, or repeatable stretchability, is often exhibited by polymers with low modulus. Brittle polymers, including those formed by polymerization of cyanoacrylates, are not generally effective in contact with biological soft tissue.

Macromers with longer distances between crosslinks are generally softer, more compliant, and more elastic. Thus, in the polymers of Hubbell, et al., increased length of the water-soluble segment, such as polyethylene glycol, tends to give more elastic gel, and these tend to adhere better, especially under stretching (as when applied to lung). Molecular weights in the range of 10,000 Daltons to 35,000 Daltons of polyethylene glycol are preferred for such applications, although ranges from 3,000 Daltons to 100,000 Daltons are useful.

An advantageous characteristic of these macromers is their ability to polymerize rapidly in an aqueous surrounding. Precisely conforming, semi-permeable, biodegradable films or membranes can thus be formed on tissue in situ to serve as biodegradable barriers, as carriers for living cells or other biologically active materials, and as surgical sealants or adhesives. In certain embodiments, the compositions are applied to the tissue and polymerized to form ultrathin coatings. This is especially useful in forming coatings on the inside of tissue lumens such as blood vessels where there is a concern regarding restenosis, and in forming tissue barriers during surgery which thereby prevent adhesions from forming.

Macromer Synthesis

The macromers for use in the compositions described herein can be synthesized using means well known to those of skill in the art. General synthetic methods are found in the literature, for example in U.S. Pat. No. 5,410,016 to Hubbell et al., U.S. Pat. No. 4,243,775 to Rosensaft et al., and U.S. Pat. No. 4,526,938 to Churchill et al., U.S. Pat. No. 6,177,095 to Sawhney et al., and U.S. Pat. No. to Pathak et al., all incorporated herein by reference in their entirety.

For example, a polyethylene glycol backbone can be reacted with trimethylene carbonate or a similar carbonate in the presence of a Lewis acid catalyst, such as stannous octoate, to form a carbonate-polyethylene glycol terpolymer. The polymer may optionally be further derivatized with additional biodegradable groups, such as lactate groups. The terminal hydroxyl groups can then be reacted with acryloyl chloride in the presence of a tertiary amine to end-cap the polymer with acrylate end-groups. Similar coupling chemistry can be employed for macromers containing other hydrophilic blocks, biodegradable blocks, and polymerizable groups.

When polyethylene glycol is reacted with carbonate and a hydroxy acid in the presence of an acidic catalyst, the reaction can be either simultaneous or sequential.

In principle, the biodegradable blocks or regions could be separately synthesized and then coupled to the backbone regions.

In synthesizing the macromer, sequential addition of biodegradable groups to a carbonate-containing macromer can be used to enhance biodegradability of the macromer after capping with polymerizable terminus.

Upon reaction of, for example, trimethylene carbonate with polyethylene glycol (PEG), the carbonate linkages in the resulting copolymers have been shown to form end linked species of PEG, resulting in segmented copolymers, i.e. PEG units coupled by one or more adjacent carbonate linkages. The length of the carbonate segments can vary, and is believed to exhibit a statistical distribution. Coupling may also be accomplished via the carbonate subunit of carbonate. These segmented PEG/carbonate copolymers form as a result of transesterification reactions involving the carbonate linkages of the carbonate segments during the carbonate polymerization process when a PEG diol is used as an initiator. Similar behavior is expected if other polyalkylene glycol initiators were used. The end-linking may begin during the reaction of the carbonate with the PEG, and completion of the end linking and attainment of equilibrium is observable by a cessation of increase of the viscosity of the solution.

If the product of this first reaction step is then reacted with a material for terminus-capping, such as acryloyl chloride, a significant percentage of the macromer end groups can be PEG hydroxyls, resulting in the attachment of the reactive groups directly to one end of a non-biodegradable PEG molecule. Such a reaction of the PEG/carbonate segmented copolymers can be prevented by adding additional segments of other hydrolyzable co-monomers (e.g. lactate, glycolate, 1,4-dioxanone, dioxepanone, caprolactone) on either end of the PEG/carbonate segmented copolymer. Some scrambling of the comonomer segments with the PEG/carbonate prepolymer is expected, but this can be minimized by using proper reaction conditions. The basic PEG/carbonate segmented copolymer or the further reacted PEG/carbonate/comonomer segmented terpolymer is then further reacted to form crosslinkable macromers by affixing each terminus with polymerizable groups (such as acrylates) to provide a macromer with reactive functionality. Subsequent reaction of the end groups in an aqueous environment results in a bioabsorbable compositions.

Macromer Compositions

The compositions of the invention can be applied to a surface in one layer in a desired thickness. In other embodiments, the applied compositions are one or more layers. The top layer or layers adhere through polymerizable groups to the bottom layer(s). This application method allows a thin coat on the surface followed by a bulk top coat of a desired thickness. The compositions for each coat can be the same or different. The polymerization can occur simultaneously or at staggered times.

The compositions as disclosed herein have increased adherence due to the adherent properties of the additive. For example in preferred embodiments, the increased adherence as measured by the following visual scoring scale:

| Score | Description |
|---|---|
| 0 | Test or control article falls off when touched or has fallen off |
| | before explant. |
| 1 | The entire test or control article can be removed by lifting one end |
| | of the application. |
| 2 | Peeling motion is required to remove application. |
| 3 | Scraping is required to remove application. |
| 4 | Vigorous, repeated scraping is required to remove application. |
| | Only be removed in pieces. |

The adherence is preferably 2.5 to 4, more preferably 3 to 4 and most preferably 3.5 to 4. The material is applied to tissue, and attempted to be removed using a surgical instrument. The determination of enhance adherence can be measured against a control composition not containing the additive by the adherence scale above or by any suitable method known in the art. For example, in the Exemplification, tissue adherence was scored using the 5-point scale (0-4, as described above) compared to control.

In certain preferred embodiments, the compositions comprising the additive and macromer are biocompatible. Compositions are considered biocompatible if the material elicits either a reduced specific humoral or cellular immune response or does not elicit a nonspecific foreign body response that prevents the material from performing the intended function, and if the material is not toxic upon ingestion or implantation. The material must also not elicit a specific reaction such as thrombosis if in contact with the blood.

Initiators for Polymerization of the Macromers

The term “initiator” is used herein in a broad sense, in that it is a composition which under appropriate conditions will result in the polymerization of a macromer. Materials for initiation may be photoinitiators, chemical initiators, thermal initiators, photosensitizers, co-catalysts, chain transfer agents, and radical transfer agents.

Photoinitiation

The initiator in certain embodiments of the invention is a photoinitiator. In discussing photoinitiators, a distinction may be drawn between photosensitizers and photoinitiators—the former absorb radiation efficiently, but do not initiate polymerization well unless the excitation is transferred to an effective initiator or carrier. Photoinitiators as referred to herein include both photosensitizers and photoinitiators, unless otherwise noted. Photopolymerizable substituents preferably include acrylates, diacrylates, oligoacrylates, dimethacrylates, or oligomethoacrylates, and other biologically acceptable photopolymerizable groups.

The formation of a hydrogel using a primer system is a three step process. The application of the primer is followed by the bulk solution and then polymerization.

The choice of the photoinitiator is largely dependent on the photopolymerizable regions. For example, when the macromer includes at least one carbon-carbon double bond, light absorption by the dye causes the dye to assume a triplet state, the triplet state subsequently reacting with the amine to form a free radical which initiates polymerization. In an alternative mechanism, the initiator splits into radical-bearing fragments which initiate the reaction. Certain dyes for use with these materials include eosin dye and initiators such as 2,2-dimethyl-2-phenylacetophenone, 2-methoxy-2-phenylacetophenone, Darocu™2959, Irgacure.™651 and camphorquinone. Using such initiators, copolymers may be polymerized in situ by long wavelength ultraviolet light or by light of about 514 nm, for example.

In certain aspects, the photoinitiator for biological use is Eosin Y, which absorbs strongly to most tissue and is an efficient photoinitiator.

It is known in the art of photopolymerization to use a wavelength of light which is appropriate for the activation of a particular initiator. Light sources of particular wavelengths or bands are well-known.

Thermal polymerization initiator systems may also be used. Systems that are unstable at 37° C. and initiate free radical polymerization at physiological temperatures include, for example, potassium persulfate, with or without tetramethyl ethylenediamine; benzoyl peroxide, with or without triethanolamine; and ammonium persulfate with sodium bisulfite. Other peroxygen compounds include t-butyl peroxide, hydrogen peroxide and cumene peroxide. As described below, it is possible to markedly accelerate the rate of a redox polymerization by including metal ions in the solution, especially transition metal ions such as the ferrous ion. It is further shown below, that a catalysed redox reaction can be prepared so that the redox-catalysed polymerization is very slow, but can be speeded up dramatically by stimulation of a photoinitiator present in the solution.

A further class of initiators is provided by compounds sensitive to water, which form radicals in its presence. An example of such a material is tri-n-butyl borane, the use of which is described below.

Redox Initiators

Metal ions can be either an oxidizer or a reductant in systems including redox initiators. For example, in some examples below, ferrous ion is used in combination with a peroxide to initiate polymerization, or as parts of a polymerization system. In this case the ferrous ion is serving as reductant. Other systems are known in which a metal ion acts as oxidant. For example, the ceric ion (4+valence state of cerium) can interact with various organic groups, including carboxylic acids and urethanes, to remove an electron to the metal ion, and leaving an initiating radical behind on the organic group. Here the metal ion acts as an oxidizer. Potentially suitable metal ions for either role are any of the transition metal ions, lanthanides and actinides, which have at least two readily accessible oxidation states. Preferred metal ions have at least two states separated by only one difference in charge. Of these, the most commonly used are ferric/ferrous; cupric/cuprous; ceric/cerous; cobaltic/cobaltous; vanadate V vs. IV; permanganate; and manganic/manganous.

Any of the compounds typically used in the art as radical generators or co-initiators in photoinitiation may be used. These include co-catalysts or co-initiators such as amines, for example, triethanolamine, as well as other trialkyl amines and trialkylol amines; sulfur compounds; heterocycles, for example, imidazole; enolates; organometallics; and other compounds, such as N-phenyl glycine.

Co-monomers can also be used such as the smaller acrylate, vinyl or allyl compounds can be used. Comonomers can also act as accelerators of the reaction, by their greater mobility, or by stabilizing radicals. Of particular interest are N-vinyl compounds, including N-vinyl pyrrolidone, N-vinyl acetamide, N-vinyl imidazole, N-vinyl caprolactam, and N-vinyl formamide.

Additional Components

Other compounds such as surfactants, stabilizers, viscosity-enhancing agents and plasticizers can be added to the initiator and/or monomer solutions. Surfactants may be included to stabilize any of the materials, either during storage or in a form reconstituted for application. Similarly, stabilizers which prevent premature polymerization may be included; typically, these are quinones, hydroquinones, or hindered phenols. Plasticizers may be included to control the mechanical properties of the final coatings. These are also well-known in the art, and include small molecules such as glycols and glycerol, sorbitol or other polyols, macromolecules such as polyethylene glycol. Other agents can be used to modulate the viscosity or elasticity of the macromer compositions to ease their application to tissue; such agents are described in U.S. Patent Publication U.S. 2002-0127196A1, incorporated herein by reference and include polysaccharides, gums and the like and preferably, hyaluronic acid.

Drugs

Biologically active materials may be included in any of the adhesive polymeric composition described herein, as ancillaries to a medical treatment (for example, antibiotics) or as the primary objective of a treatment (for example, a gene to be locally delivered). A variety of biologically active materials may be included, including passively-functioning materials such as hyaluronic acid, as well as active agents such as growth hormones. All of the common chemical classes of such agents are included. Examples of useful biologically active substances include proteins (including enzymes, growth factors, hormones and antibodies), peptides, organic synthetic molecules including antibiotics, inorganic compounds, natural extracts, nucleic acids including genes, antisense nucleotides, and triplex forming agents, lipids and steroids, carbohydrates, including hyaluronic acid and heparin, glycoproteins, and combinations thereof.

The compositions as described herein are useful for controlled drug delivery, especially of hydrophilic materials, since the water soluble regions of the polymer enable access of water to the materials entrapped within the polymer. Moreover, it is possible to polymerize the macromer containing the material to be entrapped without exposing the material to organic solvents. Release may occur by diffusion of the material from the polymer prior to degradation and/or by diffusion of the material from the polymer as it degrades, depending upon the characteristic pore sizes within the polymer, which is controlled by the molecular weight between crosslinks and the crosslink density. Deactivation of the entrapped material is reduced due to the immobilizing and protective effect of the gel and catastrophic burst effects associated with other controlled-release systems are avoided. When the entrapped material is an enzyme, the enzyme can be exposed to substrate while the enzyme is entrapped, provided the gel proportions are chosen to allow the substrate to permeate the gel. Degradation of the polymer facilitates eventual controlled release of free macromolecules in vivo by gradual hydrolysis of the terminal ester linkages.

The agents to be incorporated can have a variety of biological activities, such as vasoactive agents, neuroactive agents, hormones, anticoagulants, immunomodulating agents, cytotoxic agents, anesthetics, antibiotics, antivirals, or may have specific binding properties such as antisense nucleic acids, antigens, antibodies, antibody fragments or a receptor. Proteins including antibodies or antigens can also be delivered. Proteins are defined as consisting of 100 amino acid residues or more; peptides are less than 100 amino acid residues. Unless otherwise stated, the term protein refers to both proteins and peptides. Examples include insulin and other hormones.

Specific materials include antibiotics, antivirals, antiinflammatories, both steroidal and non-steroidal, antineoplastics, anti-spasmodics including channel blockers, modulators of cell-extracellular matrix interactions including cell growth inhibitors and anti-adhesion molecules, enzymes and enzyme inhibitors, anticoagulants and/or antithrombotic agents, growth factors, DNA, RNA, inhibitors of DNA, RNA or protein synthesis, compounds modulating cell migration, proliferation and/or growth, vasodilating agents, and other drugs commonly used for the treatment of injury to tissue. Specific examples of these compounds include angiotensin converting enzyme inhibitors, prostacyclin, heparin, salicylates, nitrates, calcium channel blocking drugs, streptokinase, urokinase, tissue plasminogen activator (TPA) and anisoylated plasminogen activator (TPA) and anisoylated plasminogen-streptokinase activator complex (APSAC), colchicine and alkylating agents, and aptomers. Specific examples of modulators of cell interactions include interleukins, platelet derived growth factor, acidic and basic fibroblast growth factor (FGF), transformation growth factor .beta. (TGF β epidermal growth factor (EGF), insulin-like growth factor, and antibodies thereto. Specific examples of nucleic acids include genes and cDNAs encoding proteins, expression vectors, antisense and other oligonucleotides such as ribozymes which can be used to regulate or prevent gene expression. Specific examples of other bioactive agents include modified extracellular matrix components or their receptors, and lipid and cholesterol sequestrants.

Examples of proteins further include cytokines such as interferons and interleukins, poetins, and colony-stimulating factors. Carbohydrates include Sialyl Lewis_{.x} which has been shown to bind to receptors for selectins to inhibit inflammation. A “Deliverable growth factor equivalent” (abbreviated DGFE), a growth factor for a cell or tissue, may be used, which is broadly construed as including growth factors, cytokines, interferons, interleukins, proteins, colony-stimulating factors, gibberellins, auxins, and vitamins; further including peptide fragments or other active fragments of the above; and further including vectors, i.e., nucleic acid constructs capable of synthesizing such factors in the target cells, whether by transformation or transient expression; and further including effectors which stimulate or depress the synthesis of such factors in the tissue, including natural signal molecules, antisense and triplex nucleic acids, and the like. Exemplary DGFE's are vascular endothelial growth factor (VEGF), endothelial cell growth factor (ECGF), basic fibroblast growth factor (bFGF), bone morphogenetic protein (BMP), and platelet derived growth factor (PDGF), and DNA's encoding for them. Exemplary clot dissolving agents are tissue plasminogen activator, streptokinase, urokinase and heparin.

Drugs having antioxidant activity (i.e., destroying or preventing formation of active oxygen) may be used, which are useful, for example, in the prevention of adhesions. Examples include superoxide dismutase, or other protein drugs include catalases, peroxidases and general oxidases or oxidative enzymes such as cytochrome P450, glutathione peroxidase, and other native or denatured hemoproteins.

Mammalian stress response proteins or heat shock proteins, such as heat shock protein 70 (hsp 70) and hsp 90, or those stimuli which act to inhibit or reduce stress response proteins or heat shock protein expression, for example, flavonoids, also may be used.

The macromers may be provided in pharmaceutically acceptable carriers known to those skilled in the art, such as saline or phosphate buffered saline. For example, suitable carriers for parenteral administration may be used.

Surfaces

Surfaces to be coated include biologically-related surfaces of all kinds. In particular, any organ, tissue or cell surface is contemplated, such as the natural surface of an organ, or a surface in a tissue created by surgical incision or wound, as well as the surface of a device or object to be used in the body or in contact with bodily fluids. In certain aspects of the invention the surface is a tissue surface of a patient (native surface). The tissue surface can also by non-native such as from a donor patient, for example, transplantation.

Moreover, the compositions described herein may be used to adhere surfaces to each other. For example, wounds in living tissue may be bonded or sealed. Medical appliances may be bonded to tissue using the compositions described herein. Examples of such applications include grafts, such as vascular grafts; implants, such as heart valves, pacemakers, artificial corneas, and bone reinforcements; supporting materials, such as meshes used to seal or reconstruct openings; and other tissue-non-tissue interfaces. Of particular interest are tissue surfaces that are friable, and therefore unable to support sutures well. Adherent polymer compositions such as those described herein can seal the suture lines, support sutured areas against mechanical stress, or substitute entirely for sutures when mechanical stress is low. Examples of such situations include vascular anastomosis, nerve repair, repair of the cornea, repair of the retina, or the cochlea, and repair of the lung, liver, kidney and spleen.

The compositions can also be used on non-tissue surfaces in general, where useful bonds may be formed between similar or dissimilar substances, and solid or gel compositions are tightly adhered to surfaces.

The compositions and methods described herein are advantageous because they can be used to coat and or to bond together any of a wide variety of surfaces. These include all surfaces of the living body, and surfaces of medical devices, implants, wound dressings and other body-contacting artificial or natural surfaces. These include, but are not limited to, at least one surface selected from the following: a surface of the respiratory tract, the meninges, the synovial spaces of the body, the peritoneum, the pericardium, the synovia of the tendons and joints, the renal capsule and other serosae, the dermis and epidermis, the site of an anastomosis, a suture, a staple, a puncture, an incision, a laceration, or an apposition of tissue, a ureter or urethra, a bowel, the esophagus, the patella, a tendon or ligament, bone or cartilage, the stomach, the bile duct, the bladder, arteries and veins; and devices such as percutaneous catheters (e.g. central venous catheters), percutaneous cannulae (e.g. for ventricular assist devices), urinary catheters, percutaneous electrical wires, ostomy appliances, electrodes (surface and implanted), and implants including pacemakers, defibrillators and tissue augmentations.

Applications for the Compositions

Methods of Treatment

Generally, any medical condition in a patient which requires a coating or sealing layer may be treated by the methods described herein to produce a coating with improved adherence over conventional means. For example, wounds may be closed; leakage of blood, serum, urine, cerebrospinal fluid, air, mucus, tears, bowel contents or other bodily fluids may be stopped or minimized; barriers may be applied to prevent post-surgical adhesions, including those of the pelvis and abdomen, pericardium, spinal cord and dura, tendon and tendon sheath. Plugs may be employed in as blocks in the fallopian tubes or as an arterial plug after the removal of catheter. The technique may also be useful for treating exposed skin, in the repair or healing of incisions, abrasions, burns, inflammation, and other conditions requiring application of a coating to the outer surfaces of the body. The technique is also useful for applying coatings to other body surfaces, such as the interior or exterior of hollow organs, including blood vessels. In particular, restenosis of blood vessels or other passages can be treated. The techniques can also be used for attaching cell-containing matrices, or cells, to tissues, such as meniscus or cartilage.

Properties

The advantages of the compositions described herein include the ability to individually determine properties of the composition that have specific properties need for the desired application. For example, certain networks are applicable to different indications where, flexibility, distensibility, firmness, uniformity and strength are desirable.

In certain circumstances, varying gelling time of the compositions disclosed herein are advantageous. For example, when a slow-gelling is wanted, it is advantageous to use system wherein the polymerization of the macromers is not instantaneous.

General Sealing of Biological Tissues

As shown in the examples below, the priming method of polymerization is especially effective in the sealing of biological tissues to prevent leakage. The examples demonstrate that a degree of sealing can be achieved with additive. The range of uses of sealing or bonding materials in the body is very large, and encompasses many potential uses. For example, in cardiovascular surgery, uses for tissue sealants include bleeding from a vascular suture line; in lung surgery, uses include stopping air leaking following resection of lobe, such as a lobectomy for removal of cancerous tissue, support of vascular graft attachment; enhancing preclotting of porous vascular grafts; stanching of diffuse nonspecific bleeding; anastomoses of cardiac arteries, especially in bypass surgery; support of heart valve replacement; sealing of patches to correct septal defects; bleeding after stemotomy; and arterial plugging. Collectively, these procedures are performed at a rate of 1 to 2 million annually. In other thoracic surgery, uses include sealing of bronchopleural fistulas, reduction of mediastinal bleeding, sealing of esophageal anastomoses, and sealing of pulmonary staple or suture lines. In neurosurgery, uses include repairs, microvascular surgery, and peripheral nerve repair. In general surgery, uses include bowel anastomoses, liver resection, kidney resection, biliary duct repair, pancreatic surgery, lymph node resection, reduction of seroma and hematoma formation, endoscopy-induced bleeding, plugging or sealing of trocar incisions, and repair in general trauma, especially in emergency procedures. In plastic surgery, uses include skin grafts, burns, debridement of eschars, and blepharoplasties (eyelid repair). In otorhinolaryngology (ENT), uses include nasal packing, ossicular chain reconstruction, vocal cord reconstruction and nasal repair. In opthalmology, uses include corneal laceration or ulceration, and retinal detachment. In orthopedic surgery, uses include tendon repair, bone repair, including filling of defects, and meniscus repairs. In gynecology/obstetrics, uses include treatment of myotomies, repair following adhesiolysis, and prevention of adhesions. In urology, sealing and repair of damaged ducts, and treatment after partial nephrectomy are potential uses. Sealing can also be of use in stopping diffuse bleeding in any of a variety of situations, including especially treatment of hemophiliacs. In dental surgery, uses include treatment of periodontal disease, tooth repair, and repair after tooth extraction. Repair of incisions made for laparoscopy or other endoscopic procedures, and of other openings made for surgical purposes, are other uses. Similar uses can be made in veterinary procedures. In each case, appropriate biologically active components may be included in the sealing or bonding materials.

Application Techniques of the Compositions and Uses

The compositions of the present invention may be administered before, during or after crosslinking. The compositions of the present invention are generally delivered to the site of administration using an apparatus that allows the components to be delivered separately. Delivery system design may allow for the mixing of the components within the device or at the application site.

Liquid spray systems would provide a very suitable means for delivering the compositions to the application site. For example, Sawhney et al., (U.S. Pat. No. 6,165,201) describe a method and device for the spray application hydrogels. The device embodies a two nozzle sprayer where the compositions are delivered from two syringes that are aerosolized by a stream of gas as the components exit the sprayer. The spray streams then merge allowing complete and even mixing in the air and at the tissue surface. Another variation of sprayer design would entail two components delivered from syringes to be mixed with a static mixer prior to delivery through a single spray nozzle.

Depending upon the properties of the components an aerosolized spray may be generated in a variety of manners including; jet, swirl nozzle, air assist, rotary and ultrasonic sprayers. Swirl nozzle technology is a common method for spraying low viscosity materials. The spray component radially enters a swirl nozzle thereby producing a tangential component to the velocity of the spray medium which produces a cone spray pattern as the medium exits the nozzle. A two component sprayer may be devised by single device that uses two syringes that are connected to two swirl nozzles. As described previously, the spray streams would then merge allowing complete and even mixing at the tissue surface. Duronio et al., (U.S. Pat. No. 6,328,229) describe a spray method using swirl nozzle technology where the two components separately and radially enter the nozzle where the mix and then are sprayed out the nozzle.

The application may also be accomplished by simple dripping of materials from a vial, bottle or syringe onto the surface to be coated. The materials may be combined within the delivery device using static mixers or could be mixed together at the application site. Application can be accomplished using common devices such as a needle, a catheter, a pipette, or a hose, depending on scale. More uniform applications may be obtained using an applicator or catheter tip, such as a brush, a pad, a sponge, a cloth, or a spreading device such as a finger, a coating blade, a balloon, or a skimming device.

Another way of delivering the compositions of the present invention is to prepare the reactive components in an inactive form (such as a liquid or powder). The composition can then be activated after application to the tissue site. The compositions described herein can be used in a variety of different applications. In general, the compositions can be adapted to use in any tissue engineering application where synthetic gel matrices are utilized such as the applications described above.

Prevention of Postoperative Adhesions

A barrier is formed as an adhesive polymeric composition for coating a surface by applying a polymerizable macromer and an additive and then polymerizing the macromer to form a network. In this application, the compositions are biodegradable and biocompatible and can be designed with selected properties of compliance and elasticity for different surfaces.

In one application, the compositions, described herein, are applied as a method of reducing formation of adhesions after a surgical procedure in a patient. The method utilizes chemical initiation or photo initiation. For example, the method includes coating damaged tissue surfaces in a patient with an aqueous solution of a free-radical polymerization initiator or redox system, a polymerizable macromer and an additive as described above.

Controlled Drug Delivery.

Another application concerns a method of locally applying a biologically active substance to tissue surfaces of a patient. The method includes the steps of mixing or covalently attaching a biologically active substance with the macromer of the compositions described herein to form a coating mixture. Tissue surfaces are coated with the mixture. The biologically active substance can be any of a variety of materials, including proteins, carbohydrates, nucleic acids, and inorganic and organic biologically active molecules. Specific examples are discussed above.

Tissue Adhesives

Another use of the compositions, described herein, is in a method for adhering tissue surfaces in a patient. The composition is applied to a tissue surface to which tissue adhesion is desired. The tissue surface is contacted with the tissue with which adhesion is desired, forming a tissue junction. The tissue junction is made when the macromers are polymerized. The additive increases the adherence of the tissues.

Tissue Coatings

In another application of these macromer compositions, a coating is applied to the surface of a tissue, for example, the lumen of a tissue such as a blood vessel. One use of such a coating is in the treatment or prevention of restenosis, abrupt reclosure, or vasospasm after vascular intervention. In one aspect, the composition is applied and then polymerized. In another aspect, the polymerization occurs as the composition is applied to the surface. Using this method, a uniform polymeric coating of between one and 500 microns in thickness can be created, for example about twenty microns, which does not evoke thrombosis or localized inflammation.

Tissue Supports

The compositions can also be used to create tissue supports by forming shaped articles within the body to serve a mechanical function. Such supports include, for example, sealants for bleeding organs, sealant in dental applications in tooth and gum repairs, sealants for bone or cartilage defects and space-fillers for vascular aneurisms. Further, such supports include strictures to hold organs, vessels or tubes in a particular position for a controlled period of time.

In certain embodiments, the compositions described herein can be used as a hydrogel. A hydrogel is a substance formed when the polymerized compositions described herein create a three-dimensional open-lattice structure which entraps water molecules to form a gel.

Packaging

The compositions materials for making the surface coatings can be packaged in any convenient way, and may form a kit including for example separate containers, alone or together with the application device. The reactive macromers are preferably stored separately from the initiator, unless they are co-lyophilized and stored in the dark, or otherwise maintained unreactive. For example, a convenient way to package the materials is in three vials (or prefilled syringes), one of which contains an additive, the second of which contains reconstitution fluid, and the third containing dry or lyophilized monomer. Dilute initiator is in the reconstitution fluid; stabilizers are in the monomer vial; and other ingredients may be in either vial, depending on chemical compatibility. An alternative packaging can be two solutions, one comprising the additive and macromer composition and the other solution containing the initiator. If the polymerization requires a two part initiator system, for example as in a redox initiation system then one part (oxidizing agent) is in the first solution and the other initiator (reducing agent) is in the second solution. In this example, the composition can be in one or both of the solutions. The additive (adherent) can be in any or all of the vials or solution depending on the method utilized. If a drug is to be delivered in the coating, it may be in any of the vials, or in a separate container, depending on its stability and storage requirements.

It is also possible, for a more “manual” system, to package some or all of the chemical ingredients in pressurized spray cans for rapid delivery. If the macromer is of low enough viscosity, for example less than 1000 cp, it can be delivered by this route. A kit might then contain a spray can of initiator; a spray can or dropper bottle of monomer, initiator and other ingredients; and an optional spreading or rubbing device. If the monomer and initiator system are designed to polymerize under the influence of natural or operating room light, possibly with the supplement of a chemical initiator or carrier such as a peroxygen compound, then the technique could be suitable for field hospital or veterinary situations.

### EXEMPLIFICATION

Macromers are often designated by a code of the form xxKZn. xxK represents the molecular weight of the backbone polymer, which is polyethylene glycol (“PEG”) unless otherwise stated, in thousands of Daltons. Z designates the biodegradable linkage, using a code wherein where L is for lactic acid, G is for glycolic acid, D is for dioxanone, C is for caprolactone, T is for trimethylene carbonate, and n is the average number of degradable groups in the block. The molecules are terminated with acrylic ester groups, unless otherwise stated. This is sometimes also indicated by the suffix A2. This code is used in the Examples below. Some specific embodiments as used in the Examples below include 1) a diacrylated multi-block copolymer of a 3.3 kDa polyethylene glycol and oligomers of lactic acid (average of 5 lactate units per macromer randomly distributed between the two ends of the polyethylene glycol) (3.3kL5A2); 2) a diacrylated multi-block copolymer of a 8 kDa polyethylene glycol and oligomers of lactic acid (average of 5 lactate units per macromer randomly distributed between the two ends of the polyethylene glycol) (8kL5A2); and 3) a diacrylated multi-block copolymer of a 20 kDa polyethylene glycol and oligomers of trimethylene carbonate and of lactic acid (average of 7 trimethylene carbonate units per macromer randomly distributed between the two ends of the polyethylene glycol followed by 5 lactate randomly distributed between the two ends of the carbonate-modified PEG) (20kT7L5A2).

For these studies, tissue adherence was scored using the 5-point scale described below:

| Score | Description |
|---|---|
| 0 | Test or control article falls off when touched or has fallen off |
| | before explant. |
| 1 | The entire test or control article can be removed by lifting one |
| | end of the application. |
| 2 | Peeling motion is required to remove application. |
| 3 | Scraping is required to remove application. |
| 4 | Vigorous, repeated scraping is required to remove application. |
| | Only be removed in pieces. |

### Example 1

Adherence of hydrogel formulations were scored using the pig myocardium model, in vivo beating heart (atria and ventricles). Lapse of time between the gel deposition and adherence scoring was 2 hours. The number of application sites per formulation was 2.

The experiment was conducted on two test groups: a control and treatment groups described below. The effect of PEG-dialdehyde as a tissue-reactive additive was evaluated by comparing the tissue adherence of a macromer solution (Solution 1) overlayed with a macromer overcoat or top layer (Overcoat 1) with a Solution 1 containing 10% 10 kDa PEG-dialdehyde (Solution 2) overlayed with Overcoat 1. Compositions of Solution 1, Solution 2 and Overcoat 1 are shown in Table A. Using an in vivo, beating pig heart model, Solution 1 and Solution 2 were brushed onto separate sites of the heart. Overcoat 1 was then mixed into each of the solution sites. Overcoat 1 was then applied in excess and both sites were then illuminated with visible light (100 mW/cm², 40 seconds) to initiate photopolymerization.

TABLE A
Compositions used to test PEG-dialdehyde as tissue-reactive additive

| Solution 1 | Solution 2 | Overcoat 1 |
|---|---|---|
| 30% | 30% 3.3kL5A2 macromer | 10% 20kTLA2 macromer |
| 3.3kL5A2 | | |
| macromer | | |
| 3% sodium | 3% sodium chloride | 0.54% triethanolamine |
| chloride | | |
| 2000 ppm | 2000 ppm eosin-Y | 0.8% potassium phosphate, |
| eosin-Y | | monobasic |
| 0.5% ferrous | 0.5% ferrous gluconate | 0.4% N-vinyl caprolactam |
| gluconate | | |
| 1% fructose | 1% fructose | 0.0125% tert-butyl |
| | | hydroperoxide |
| in water | 10% 10 kDa PEG-dialdehyde | 0.004% eosin-Y |
| for injection | in water for injection | in water for injection |

After 2 hours, the gels were evaluated for tissue adherence using the 5-point score system described above. The results are shown in Table 1. Results showed that the gel without additive (Solution 1) could be removed by lifting one end of the application and left no residual gel on the tissue. In contrast, gels containing the additive (Solution 2) required peeling and scraping to remove the gel and residual gel remained on the tissue after scraping.

TABLE 1
Tissue adherence results on a beating pig heart demonstrating
PEG-dialdehyde as tissue-reactive additive

| | Adherence | |
|---|---|---|
| Formulation | Scores | Comments |
| Solution 1 + Overcoat 1 | 1, 1 | Gels could be removed by lifting |
| | | one end of the application and left |
| | | no residual gel on the tissue. |
| Solution 2 + Overcoat 1 | 2.5, 2.5 | Gels showed improved adherence |
| | | over control gels with peeling and |
| | | scraping was required to remove |
| | | the gel. Residual gel remained |
| | | on the tissue after scraping. |

### Example 2

The effect of PEG-disuccinimidyl glutarate (Sun Bio West, Orinda, Calif.) was evaluated as a tissue-reactive additive by comparing gels prepared using Solution 1 and Overcoat 1 with gels prepared with Solution 1 containing 10% 8 kDa PEG-disuccinimidyl glutarate (Solution 3) and Overcoat 1. The composition of Solution 3 is detailed in Table B.

TABLE B
Composition used to test PEG-disuccinimidyl glutarate
as tissue-reactive additive
Solution 3

30% 3.3kL5A2 macromer
3% sodium chloride
2000 ppm eosin-Y
0.5% ferrous gluconate
1% fructose
10% 8 kDa PEG-disuccinimidyl glutarate
in water for injection

On separate sites on an in vivo, beating pig heart model, Solution 1 and Solution 3 were brushed into the myocardium. Over each of the solution sites, Overcoat 1 was mixed into the solution sites and then an excess of Overcoat 1 was then applied over the sites. Each of the gel sites was illuminated with visible light (100 mW/cm², 40 seconds) to initiate photopolymerization. After 5 minutes, gels were scored for tissue adherence using the 5-point scoring system described above. The results are shown in Table 2. Results from this study showed that gels prepared with Solution 1 and Overcoat 1 required peeling and scraping to remove the gels. Gels containing the tissue-reactive additive (Solution 3) showed improved adherence as peeling, scraping and repeated scraping was required to remove the gels.

TABLE 2
Tissue adherence results on a beating pig heart demonstrating 8 kDa
PEG-disuccinimidyl glutarate as tissue-reactive additive

| | Adherence | |
|---|---|---|
| Formulation | Scores | Comments |
| Solution 1 + Overcoat 1 | 2.0, 2.5 | Peeling motion was required |
| | | to remove first application. Peeling |
| | | and scraping was required to |
| | | remove second application. |
| Solution 3 + Overcoat 1 | 2.5, 3.5, | In general, gels showed |
| | 4.0 | improved adherence over control |
| | | gels with peeling, scraping and |
| | | repeated, vigorous scraping |
| | | required to remove the gels. |

### Example 3

The effect of PEG-disuccinimidyl glutarate as a tissue-reactive additive was evaluated by comparing the tissue adherence of a macromer solution (Solution 1) overlayed with an macromer overcoat (Overcoat 1) with Solution 3 overlayed with Overcoat 1. Using an in vivo, non-beating pig myocardium model, Solution 1 and Solution 3 were brushed onto separate sites of the heart. Overcoat 1 was then mixed into each of the solution sites. Overcoat 1 was then applied in excess and both sites were then illuminated with visible light (100 mW/cm², 40 seconds) to initiate photopolymerization.

After 5 minutes, gels were scored for adherence using the 5-point scoring scale described above as shown in Table 3). Gels prepared with Solution 1 required light to moderate scraping to be removed from the tissue. Gels prepared with Solution 3 showed improved adherence over Solution 1 gels with repeated, vigorous scraping required to remove the gels from the myocardium.

TABLE 3
Tissue adherence results on a non-beating pig heart demonstrating 8 kDa
PEG-disuccinimidyl glutarate as tissue-reactive additive

| | Adherence | |
|---|---|---|
| Formulation | Scores | Comments |
| Solution 1 + Overcoat 1 | 3.5, 3 | Gels required light to moderate |
| | | scraping to be removed from the |
| | | tissue. |
| Solution 3 + Overcoat 1 | 4, 4 | Gels showed improved |
| | | adherence over control gels |
| | | with repeated, vigorous scraping |
| | | required to remove the applications |
| | | from the tissue. |

### Example 4

The effect of PEG-disuccinimidyl glutarate (Sun Bio West, Orinda, Calif.) was evaluated as a tissue-reactive additive by comparing gels prepared using Solution 1 and Overcoat 1 with gels prepared with Solution 3 containing 10% 8 kDa PEG-disuccinimidyl glutarate and Overcoat 1. On separate sites on intact pig dura, Solution 1 and Solution 3 were brushed into the tissue. Over each of the solution sites, Overcoat 1 was mixed into the solution sites and then an excess of Overcoat 1 was then applied over the sites. Each of the gel sites was illuminated with visible light (100 mW/cm², 40 seconds) to initiate photopolymerization.

After 5 minutes, gels were scored for tissue adherence using the 5-point scoring system described above and shown in Table 4. Results from this study showed that gels prepared with Solution 1 and Overcoat 1 could be removed by scraping the tissue. Gels containing the tissue-reactive additive (Solution 3) showed improved adherence over Solution 1 with more forceful scraping required to remove the gel from the tissue.

TABLE 4
Tissue adherence results on intact pig dura demonstrating 8 kDa PEG-
disuccinimidyl glutarate as tissue-reactive additive

| | Adherence | |
|---|---|---|
| Formulation | Scores | Comments |
| Solution 1 + Overcoat 1 | 3 | Gels could be removed by |
| | | scraping the tissue. |
| Solution 3 + Overcoat 1 | 3.5 | Gels showed improved |
| | | adherence over the control with |
| | | more forceful scraping required to |
| | | remove the gel from the tissue. |

Examples 1-4 show the tissue adherence of Sealant formulations improved with the addition of an additive (PEG-dialdehyde and PEG-disuccinimidyl glutarate).

### Example 5

The following experiment demonstrates sprayable formulations to form hydrogels that set up quickly, adhere to tissue, and are potentially suitable for drug delivery and adhesion barrier applications. The materials employ redox chemistry for radical initiation of acrylated macromer formulations and PEG-dialdehydes to promote tissue adhesion.

Ferrous gluconate (FeGlu)/tert-butylhydroperoxide (t-BHP) was chosen as the radical initiation system to crosslink diacrylate-terminated PEG macromers to form the bulk of the hydrogel network. Reducing and oxidizing solutions were prepared separately and delivered from dual syringes for spraying applications. The PEG dialdehyde were added to both of the reducing and oxidizing solutions. One or both of the aldehydes on the PEG chain may covalently bind to primary amines on the tissue and adhere the hydrogel to the tissue through polymer chain entanglement.

Design of Experiment (DOE)

Methods: Synthesis of the modified PEG macromers used in this study has been described in Pathak, C P., et al., Macromolecules, 26, 581-587 (1993). PEG dialdehydes were acquired from SunBiowest (Orinda, Calif.). Vinyl caprolactam (VC, Charkit Chem., Darien, Conn.) was distilled prior to use. Teriary-butyl hydroperoxide (T-BHP) (Acros Chemicals, N.J.), triethanolamine (TEOA) and Ferrous gluconate (FeGlu) were acquired from Spectrum (Gardena, Calif.) and used as received. Deionized water was used as the diluent for all solutions. All components and stock solutions were stored at −20° C. prior to use. To simplify sample preparation, stock solutions of some ingredients were prepared as follows:
1. T-BHP stock solution=0.063 g (70% t-BH in water) in 50.174 g deionized water.
2. TEOA stock solution=0.6237 g TEOA into 50.0897 g deionized water. This solution was adjusted to pH-7 with 2N HCl prior to final weight adjustments. Final pH=7.04.
3. FeGluconate stock solution=1.5023 g dissolved in 50.082 g deionized water

All stock solutions were stored at −20° C. and thawed just prior to use.

The selection of the components for the hydrogel formulations and their concentration levels considered are shown in Table C. A seven factor, three level design of experiments was developed to screen 27 final hydrogel compositions out of 2187 potential combinations of these components. The compositions of the actual 27 hydrogel formulations were prepared from the variable components as identified in Table 6. For each hydrogel composition 1 to 27 of Table 6, a reducing solution and an oxidizing solutions were identical within each pair but for the presence of either the reducing reagent in the reducing solution or oxidizing reagent in the oxidizing solution, as exemplified for compositions No. 13 in Table D. The individual components for each pair of reducing and oxidizing solution were varied from one pair to another in accordance with variables as selected in Table 6.

TABLE C
Levels used for each factor in the experimental design.

| | Reducing solution | Oxidizing solution |
|---|---|---|
| PEG Macromers, Mw | 3.3kL5A2, 8k L5A2, | 3.3k kL5A2, 8k kL5A2, |
| | 20 L5A2k | 20kL5A2k |
| PEG aldehydes, Mw | 3.4k, 8k, 10k | 3.4k, 8k, 10k |
| PEG Macromer, | 5, 10, 15 | 5, 10, 15 |
| w/w % | (0.05, 0.1, 0.15) | (0.05, 0.1, 0.15) |
| (g/g of solution) | | |
| PEG aldehyde, w/w | 5, 10, 15 | 5, 10, 15 |
| (g/g of solution) | (0.05, 0.1, 0.15) | (0.05, 0.1, 0.15) |
| t-BHP, ppm | 0 | 62.5, 125, 250 |
| (g t-BHP stock/g of | | (0.05, 0.1, 0.2) |
| solution) | | |
| FeGlu, w/w % | 0.6 | 0 |
| (g FeGlu stock/g of | (0.2 g) | |
| solution) | | |
| TEOA, w/w % | 0, 0.25, 0.5 | 0, 0.25, 0.5 |
| (g stock/g of | (0, 0.2, 0.4) | (0, 0.2, 0.4) |
| solution) | | |
| VC, mg/g of solution | 5, 10, 15 | 5, 10, 15 |

Table C also shows the mass of stock solutions used per gram of reducing or oxidizing solution. The PEG macromers and aldehydes were added as powders. VC (Lot # 991103) was melted in a sealed vial and then added by pipetting. The balance of each gram of either oxidizing or reducing solution was made up of deionized water. For example, composition #13 in Table 6 was prepared from an oxidizing solution and a reducing solution as shown in Table D.

TABLE D
Amounts of components in the redox pairs (Reduction solution/oxidixing
solution) used to make Composition No. 13

| | Reducing | |
|---|---|---|
| | Solution | Oxidizing solution |
| 8kL5A2 PEG | 0.2 | 0.2 |
| Macromers, g | | |
| 8k PEG dialdehyde, g | 0.2 | 0.2 |
| TEOA stock, g | 0.8 | 0.8 |
| VC, g | 0.03 | 0.03 |
| FeGluc stock, g | 0.4 | 0 |
| t-BHP stock, g | 0 | 0.4 |
| DI water | 0.37 | 0.37 |
| Total, g | 2.0 | 2.0 |

The viscosity of each reducing solution was measured with a Bohlin rheometer (1.5 ml, 150 um gap, CP4/40 spindle, 0.08-200 s−1 shear rate, 25° C.). Gelation times were determined by pipetting 100 ul of each solution onto a spinning 8 mm stir bar and timing when the magnet stopped moving. The resulting gel was scored 0-4 on the basis of firmness:
0=liquid
1=very weak gel, it will hang in a blob from tweezers
2=soft gel, it droops when held with tweezers, easily crushed with fingers
3=firm gel, it keeps its shape in tweezers, takes some pressure to crush
4=very firm gel, keeps its shape, is difficult to crush
   and uniformity:
0=75-100% liquid, by eye
1=50-75% liquid, by eye
2=25-50% liquid, by eye
3=10-25% liquid, by eye
4=0-10% liquid, by eye

Gelation on porcine pericardium was conducted in a similar manner and adhesion of gel to tissue was scored according to the adherence 5-point scale as described earlier. Of the 27 samples in Table 6 and Table 7, three scored higher than 10.0 on the combined score of gel firmness, uniformity, and adhesion. Compositions No. 13 and No. 27 had viscosities lower than those thought to be necessary for spraying with a handheld sprayer (<˜30 cP). Composition No. 13 was chosen for further development because it had the best combination of low viscosity and high adherence.

Experiment 5B: Evaluation of Sprayability and Tissue Adhesion.

Methods: Composition No. 13 and an aldehyde-free control version of No. 13 were selected for spraying onto porcine pericardium. 2-5 g of each reducing and oxidizing spray solutions were prepared according to Tables E and F below.

TABLE E
Aldehyde-containing Composition No. 13 for spraying experiment.

| | Reducing Solution | Oxidizing solution |
|---|---|---|
| 8kL5A2 PEG | 0.2009 | 0.2019 |
| Macromer, g | | |
| 8k PEG dialdehyde, g | 0.2002 | 0.2009 |
| TEOA stock, g | 0.8 | 0.8 |
| VC, g | 0.03 | 0.03 |
| FeGluc stock, g | 0.4 | 0 |
| t-BHP stock, g | 0 | 0.4 |
| DI water | 0.368 | 0.368 |

TABLE F
Aldehyde-free Composition No. 13 for spraying experiment

| | Reducing | |
|---|---|---|
| | Solution | Oxidizing solution |
| 8kL5A2 PEG | 0.5040 | 0.5040 |
| Macromer, g | | |
| 8k PEG (Clariant, | 0.5024 | 0.5038 |
| Basel, Switzerland), g | | |
| TEOA stock, g | 2.0 | 2.0 |
| VC, g | 0.075 | 0.075 |
| FeGluc stock, g | 1.0 | 0 |
| t-BHP stock, g | 0 | 1.0 |
| DI water | 0.920 | 0.92 |

A double barreled handheld syringe sprayer was used to deliver the reducing and oxidizing solutions simultaneously to the pericardium site. Gel adherent to the pericardium was scored before and after soaking in PBS overnight at room temperature.

Results

The reducing and oxidizing solutions combined in the mist produced by the two spray streams of the handheld sprayer and immediately gelled onto a vertical piece of porcine pericardium approximately 2-3 cm away from the nozzle. The resulting gel scored a 3.5 in the adhesion scoring system outlined previously. Pieces that were soaked on the pericardium remained well adherent the next day (Score=3). The control composition that contained aldehyde-free PEG did not adhere as well to the pericardium and could be removed in large pieces (score=2).

This experiment demonstrated that modified PEG-macromers can be used to create solutions that are inviscid enough to be sprayed, but concentrated enough to rapidly form a stable gel on a vertical surface. Polymer entanglement of tissue-bound PEG-aldehydes with the macromers is also shown to effectively adhere gels to tissues. This experiment demonstrates that these materials may be suitable as surgical adhesion barriers, tissue sealants, and drug delivery vehicles.

TABLE 6
Composition Formulation

| | 2 | 3 | | 5 | | | | |
|---|---|---|---|---|---|---|---|---|
| 1 | Macromer | Macromer | 4 | Aldeyde | 6 | | | |
| ID | MW | Conc. | Aldeyde | conc. | VC | 7 | 8 | 9 |
| No. | Da | W % | MW Da | w % | w % | TBH ppm | TEOA w % | μ, cP |
| 1 | 3.3K | 5 | 10K | 10 | 15 | 125 | 0 | 11.0 |
| 2 | 20K | 10 | 10K | 5 | 10 | 250 | 0 | 26.5 |
| 3 | 20K | 15 | 3.4K | 10 | 5 | 125 | 0.5 | 63.1 |
| 4 | 3.3K | 10 | 3.4K | 15 | 15 | 125 | 0 | 16.2 |
| 5 | 8K | 5 | 3.4K | 5 | 5 | 62.5 | 0 | 4.6 |
| 6 | 8K | 15 | 10K | 15 | 10 | 125 | 0.25 | 76.5 |
| 7 | 20K | 10 | 10K | 5 | 15 | 62.5 | 0.25 | 25.5 |
| 8 | 8K | 15 | 10K | 15 | 5 | 62.5 | 0 | 75.5 |
| 9 | 8K | 10 | 8K | 10 | 10 | 125 | 0.25 | 23.2 |
| 10 | 8K | 5 | 3.4K | 5 | 15 | 250 | 0.5 | 4.7 |
| 11 | 3.3K | 15 | 8K | 5 | 15 | 125 | 0 | 12.6 |
| 12 | 8K | 15 | 10K | 15 | 15 | 250 | 0.5 | 80.2 |
| 13 | 8K | 10 | 8K | 10 | 15 | 250 | 0.5 | 28.1 |
| 14 | 8K | 5 | 3.4K | 5 | 10 | 125 | 0.25 | 4.5 |
| 15 | 3.3K | 5 | 10K | 10 | 5 | 250 | 0.25 | 10.5 |
| 16 | 20K | 5 | 8K | 15 | 10 | 250 | 0 | 28.3 |
| 17 | 20K | 10 | 10K | 5 | 5 | 125 | 0.5 | 24.0 |
| 18 | 20K | 15 | 3.4K | 10 | 15 | 62.5 | 0.25 | 69.5 |
| 19 | 8K | 10 | 8K | 10 | 5 | 62.5 | 0 | 21.3 |
| 20 | 20K | 5 | 8K | 15 | 15 | 62.5 | 0.25 | 28.9 |
| 21 | 3.3K | 15 | 8k | 5 | 5 | 250 | 0.25 | 13.0 |
| 22 | 3.3K | 10 | 3.4K | 15 | 10 | 62.5 | 0.5 | 15.2 |
| 23 | 20K | 15 | 3.4K | 10 | 10 | 250 | 0 | 63.7 |
| 24 | 3.3K | 5 | 10K | 10 | 10 | 62.5 | 0.5 | 14.3 |
| 25 | 3.3K | 15 | 8K | 5 | 10 | 62.5 | 0.5 | 14.0 |
| 26 | 20K | 5 | 8K | 15 | 5 | 125 | 0.5 | 27.5 |
| 27 | 3.3K | 10 | 3.4K | 15 | 5 | 250 | 0.25 | 15.3 |

TABLE 7
Evaluation of the Compositions from Table 6

| 1 | 10 | 11 | 12 | 13 | 14 |
|---|---|---|---|---|---|
| ID No. | Gel time, s | Firmness | Uniformity | Adherence | Sum |
| 1 | 3 | 0 | 0 | 0 | 0 |
| 2 | 16 | 0 | 0 | 3.5 | 3.5 |
| 3 | 15 | 2 | 3 | 3 | 8 |
| 4 | 3 | 3 | 3 | 1 | 7 |
| 5 | >120 | 0 | 0 | 0 | 0 |
| 6 | 4 | 3 | 2 | 2.5 | 7.5 |
| 7 | 8 | 2.5 | 2 | 2 | 6.5 |
| 8 | 6 | 2 | 2 | 0 | 4 |
| 9 | 5 | 3 | 3 | 3 | 9 |
| 10 | 2 | 3 | 3 | 2 | 8 |
| 11 | 3 | 2.5 | 3 | 1 | 6.5 |
| 12 | 2 | 3 | 4 | 3.5 | 10.5 |
| 13 | 2 | 4 | 3 | 3.5 | 10.5 |
| 14 | 7 | 2 | 3 | 0 | 5 |
| 15 | 3 | 1 | 2 | 1 | 4 |
| 16 | 12 | 0.5 | 1 | 1.5 | 3 |
| 17 | 6 | 2 | 3 | 2 | 7 |
| 18 | 10 | 2.5 | 2 | 1.5 | 6 |
| 19 | 10 | 1 | 1 | 3.5 | 5.5 |
| 20 | 9 | 1 | 1 | 0.5 | 2.5 |
| 21 | 3 | 3.5 | 3 | 1 | 7.5 |
| 22 | 5 | 3 | 2 | 0 | 5 |
| 23 | 6 | 2.5 | 3 | 2 | 7.5 |
| 24 | 6 | 2 | 1 | | 3 |
| 25 | 4 | 1 | 1 | 0 | 2 |
| 26 | 6 | 1 | 1 | 0 | 2 |
| 27 | 3 | 4 | 4 | 3 | 11 |

Column Legend:
10. Gelation time of product by stir-bar test, seconds
11. Firmness of gel product score, 0-4 (0 = liquid, 4 = very firm)
12. Uniformity of gel product score, 0-4 (0 = mostly liquid, 4 = mostly gel)
13. Adherence of gel product to porcine pericardium score, by stir-bar test (0 = no adherence, 4 = strong adherence)
14. Sum of firmness, uniformity, and adherence scores.

While this invention has been particularly shown and described with references to preferred embodiments thereof, it will be understood by those skilled in the art that various changes in form and details may be made therein without departing from the scope of the invention encompassed by the appended claims.

1. An adhesive polymeric composition, comprising: a polymerized macromer network and an additive mixed or entangled in the polymerized macromer network, wherein the additive is bonded to a surface by at least one covalent bond or by secondary interactions, wherein the additive is not covalently bonded to the polymerized macromer network.

2. The composition of claim 1, wherein the additive is covalently bonded to the surface by at least one covalent bond, wherein the covalent bond is formed by the reaction between a functional group (hereinafter “surface reactive group”) on the additive and a functional group on the surface (hereinafter additive reactive group”).

3. The composition of claim 2, wherein the additive is a polymer bonded to the surface through a covalent bond formed by a reaction between a surface reactive group at each terminus and an additive reactive group.

4. The composition of claim 2, wherein the surface reactive group is an aldehyde, N-hydroxysuccinimide ester and related active esters, maleimide, isocyanate, disulfide, epoxide, aziridine, carbodiimide, episulfide, ketene, carboxylate, phosphate, alkyl halides (alkylating agents) or alkyl sulfonate esters (alkylating agents).

5. The composition of claim 1, wherein the surface is a tissue surface of a patient.

6. The composition of claim 1, wherein the composition is biodegradable.

7. The composition of claim 1, wherein the composition further comprises a drug.

8. The composition of claim 7, wherein the drug is covalently attached to the polymerized macromer network.

9. The composition of claim 1, wherein the polymerized macromer network comprises a biodegradable block and a hydrophilic block between two free radical polymerizable groups.

10. The composition of claim 1, wherein the macromer comprises a copolymer core with an acrylate group at each terminus.

11. The composition of claim 9, wherein the biodegradable block of the macromer is selected from a carbonate (trimethylene carbonate), an ester (lactate, glycolate, hydroxyethylglycolate (the open ester of dioxanone)), or an orthoester.

12. The composition of claim 10, wherein the copolymer core is polyethylene glycol-multi-block copolymer including a biodegradable block.

13. The composition of claim 12, wherein the composition further comprises a biodegradable block of trimethylenecarbonate and/or lactate.

14. The composition of claim 1, further comprising a top layer of a second polymerized macromer wherein the first polymerized macromer is bonded to the second polymerized macromer.

15. An adhesive polymer composition comprising a polymerized macromer network and an additive mixed or entangled in the polymerized macromer, wherein: the additive is bonded to a surface by at least one covalent bond formed by the reaction between a functional group on the additive (hereinafter “Functional Group A”) and a functional group on the surface; and the additive is bonded to the macromer network by at least one covalent bond formed by the reaction between a functional group on the additive (hereinafter “Functional Group B”) and a functional group on the macromer network; wherein functional group A is non-reactive with the macromer network and Functional Group B is non-reactive with the surface.

16. The composition of claim 15, wherein the surface is a tissue surface of a patient.

17. The composition of claim 15, wherein the composition is biodegradable.

18. The composition of claim 16, wherein the functional group A of the additive is selected from an aldehyde, N-hydroxysuccinimide ester and related active esters, maleimide, isocyanate, disulfide, epoxide, aziridine, carbodiimide, episulfide, ketene, carboxylate, phosphate, alkyl halides (alkylating agents) and alkyl sulfonate esters (alkylating agents).

19. The composition of claim 15, wherein functional group B is a free radical polymerizable group.

20. The composition of claim 15, further comprising a drug.

21. The composition of claim 20, wherein the drug is covalently attached to one of the macromers.

22. The composition of claim 15, wherein the polymerized macromer network comprises a biodegradable block and a hydrophilic block between two free radical polymerizable groups.

23. The composition of claim 15, wherein the macromer comprises a copolymer core with an acrylate group at each terminus.

24. The composition of claim 22, wherein the biodegradable block of the macromer is selected from one or more of a carbonate (trimethylene carbonate), an ester or (lactate, glycolate, hydroxyethylglycolate (the open ester of dioxanone), or an orthoester.

25. The composition of claim 23, wherein the copolymer core is polyethylene glycol multi-block copolymer including a biodegradable block.

26. A polymeric composition for adhering to a surface, comprising:
a) a first solution of at least one polymerizable macromer;
b) an additive having at least one surface reactive group and wherein the additive is non reactive with the macromer; and
c) a polymerization initiator or a first and second agent that when combined reacts to form a polymerization initiator;
wherein polymerization of the macromer of a) is initiated by the initiator of c).

27. The composition of claim 26, wherein the polymerization initiator is a redox or free-radical initiator.

28. The composition of claim 26, wherein the additive comprises a surface reactive group at each terminus that can bind to the surface covalently or through secondary interaction.

29. The composition of claim 26, wherein the additive comprises a surface reactive group at each terminus that can bind to the surface covalently.

30. The composition of claim 26 wherein the surface reactive group is an aldehyde, N-hydroxysuccinimide ester and related active esters, maleimide, isocyanate, disulfide, epoxide, aziridine, carbodiimide, episulfide, ketene, carboxylate, phosphate, alkyl halides (alkylating agents) or alkyl sulfonate esters (alkylating agents).

31. The composition of claim 26 further comprising a drug.

32. The composition of claim 31, wherein the drug is covalently attached to the macromer.

33. The composition of claim 26, wherein the macromer comprises a hydrophilic block between two free radical polymerizable groups and a biodegradable block.

34. The composition of claim 26, wherein the macromer comprises a copolymer core with an acrylate group at each terminus.

35. The composition of claim 32, wherein the biodegradable block of the macromer is selected from one or more of a carbonate (trimethylene carbonate), an ester (lactate, glycolate, hydroxyethylglycolate (the open ester of dioxanone)), or an orthoester.

36. The composition of claim 34, wherein the copolymer core is polyethylene glycol multi-block copolymer including a biodegradable block.

37. The composition of claim 26, additionally comprising a second solution of the second agent and wherein the first agent is dissolved in the first solution.

38. The composition of claim 37, wherein the first and second agent form a redox-based free-radical-generating system when combined.

39. The composition of claim 38, wherein the second solution comprises at least one polymerizable macromer polymerizable with the macromer of the first solution.

40. The composition of claim 36, wherein at least one surface reactive group of the additive covalently bonds to the surface.

41. A polymeric composition for adhering to a surface, comprising
a) a polymerizable macromer,
b) an additive comprising at least one Functional Group A and at least one Functional Group B, wherein Functional Group A can react with a functional group on the surface to form a covalent bond with the surface but is non-reactive with the polymerizable macromer and wherein Functional Group B can form a covalent bond with a functional group on the polymerizable macromer to form a covalent bond with the polymerizable macromer (or polymerized macromer) but is non-reactive with the surface; and
c) a polymerization initiator or a first and second agent that when combined reacts to form a polymerization initiator, wherein polymerization of the macromer of a) is initiated by the initiator of c).

42. A method of coating a surface, comprising:
a) applying to the surface an additive capable of binding to the surface and a polymerizable macromer;
b) binding said additive to the surface; and
c) polymerizing said macromer, thereby entangling the additive with the polymerized macromer.

43. The method of claim 42, wherein the additive binds covalently to the surface.

44. The method of claim 42, wherein the macromer and additive are simultaneously applied to the surface.

45. The method of claim 42 where a macromer or additive is applied to the surface prior to step a) wherein the macromer and additive being simultaneously applied the surface.

46. The method of claim 42 where an additional macromer or additive is applied to the surface prior to step a) wherein the macromer and additive are simultaneously applied to the surface.

47. The method of claim 42, wherein polymerization of the macromer is initiated prior to applying to the surface.

48. The method of claim 42, wherein polymerization is initiated while the macromer is being applied to the surface.

49. The method of claim 42, wherein polymerization is initiated after applying the macromer to the surface.

50. The method of claim 42, wherein the additive is a polymer that binds to the surface through a covalent bond at one or more terminus.

51. The method of claim 42, wherein the surface is a tissue surface of a patient.

52. The method of claim 42, wherein the additive and the macromer are added together and a top coat of a second polymerizable macromer is applied after step b) and polymerized.

53. A method of adhering a polymeric composition to a surface, comprising:
a) applying an additive and a polymerizable macromer to the surface, wherein the additive comprises a Functional Group A and a Functional Group B, wherein Functional Group A can react with a functional group on the surface to form a covalent bond with the surface but is non-reactive with the polymerizable macromer and wherein Functional Group B can form a covalent bond with a functional group on the polymerizable macromer to form a covalent bond with the polymerizable macromer (or polymerized macromer) but is non-reactive with the surface;
b) polymerizing the macromer to form a polymerized macromer network; and
c) reacting Functional Group A with the surface, reacting Functional Group B with the polyermizable macromer or polymerized macromer network.
